(19) United States
(12) Patent Application Publication
(10) Pub. No.: US 2004/0193084 A1
(43) Pub. Date: Sep. 30, 2004

(54) Vein compressor device

(75) Inventor: Sundaram Ravikumar, Briarcliff Manor, NY (US)

Correspondence Address: GORDON & JACOBSON, P.C., 65 WOODS END ROAD, STAMFORD, CT, 06905, US

(73) Assignee: ARVIK ENTERPRISES, LLC

(21) Appl. No.: 10/400,901

(22) Filed: Mar. 27, 2003

(51) Int. Cl. A61F 5/00

(52) U.S. Cl. 602/13

(57) ABSTRACT

A device is provided for applying pressure to the human leg for use in conjunction with treatment of varicose veins. The device includes a flexible member and at least one air bladder chamber integral thereto that are adapted to securely wrap around the human leg. A tube in fluid communication with the air bladder chamber(s) extends to an air pumping mechanism that operates to inflate the air bladder chamber(s) to a pressurized state. The flexible member preferably includes an opening at the knee joint level to enable a patella to protrude therethrough. In addition, the flexible member preferably extends below knee joint level and is adapted to securely wrap around a lower portion of a leg to provide stability to the leg. Preferably, the air bladder chamber of the device is substantially longer in a first dimension than in a second dimension orthogonal thereto such that the air bladder chamber can be positioned to cover a portion of the human leg that is relatively long in the vertical dimension and narrow in the horizontal dimension.

### BACKGROUND OF THE INVENTION

1. Field of the Invention

This invention relates to medical apparatus. More particularly, this invention relates to mechanisms for applying pressure to a lower limb of the human body in conjunction with treatment of varicose veins in the lower limb.

2. State of the Art

Arterial blood flows through the main axial arteries to the upper and lower limbs. It returns via the deep and superficial veins. The veins in the lower limbs are typically divided into three categories: superficial veins which run near the surface and return blood from cutaneous structures; deep veins which run within deep chambers and muscles and follow arteries and are named for their companion arteries; and communicating (also called perforating) veins which connect the superficial veins to the deep veins.

In the lower limb, the superficial veins carry only about 10% of the blood, while the remainder passes via the deep veins. The superficial veins lie superficial to the muscle fascia of the limb. The principal superficial veins in the leg are the long and short saphenous veins. The short saphenous vein terminates at the saphenopopliteal junction (SPJ) and the long saphenous vein at the saphenofemoral junction (SFJ) in the groin. Here the flow in the superficial veins joins that in the deep veins. There is, in addition, a number of places in the calf and thigh where flow in the superficial veins may also join that in the deep veins. In the ankle, calf and thigh, communicating veins connect the superficial to the deep venous system. Near the ankle are the Cockett perforating veins, near the knee the Boyd perforators, and in the thigh the Hunterian perforating vein. All veins in the upper and lower limbs contain valves every few centimeters which ensure that blood flows in one direction only; i.e., towards the heart.

The venous valves are usually bicuspid valves, with each cusp forming a sack or reservoir for blood which, under pressure, forces the free surfaces of the cusps together to prevent retrograde flow of the blood and allow antegrade flow to the heart. An incompetent valve is a valve which is unable to close because the cusps do not form a proper seal and retrograde flow of blood cannot be stopped.

The varicose vein condition includes dilatation and tortuosity of the superficial veins of the lower limb, resulting in unsightly discoloration, pain and ulceration. Varicose veins often involve incompetence of one or more venous valves, which allow reflux of blood from the deep venous system to the superficial venous system or reflux within the superficial system.

Varicose veins are compatible with long life and rarely cause fatal complications, but the condition significantly decreases the quality of life. Patients complain primarily of leg fatigue, dull, aching pains, ankle swelling, and ulcerations. Occasionally, thrombosis occurs in dilated subcutaneous channels, resulting in local pain, induration, edema, inflammation, and disability. In addition to those problems, the high visibility of the unattractive rope-like swellings and reddish skin blotches causes considerable distress for both men and women. Lastly, varicose eczema, which is a local reddened swollen and itching skin condition can occur and can spread to distant parts of the body (called an “Id reaction”).

A common technique employed in the treatment of varicose veins involves surgical ligation and stripping of the problem vein. When treating the great saphenous vein, this procedure typically involves making a 2.5 cm incision in the groin to expose the sapheno-femoral junction, where the great saphenous vein and its branches are ligated with a heavy ligature. The distal portion of the vein is exposed, and a flat metal or plastic stripper is introduced to exit in the proximal saphenous vein. The leg is held vertically to empty the venous tree before stripping the vein from the ankle to the groin. If the small saphenous vein is also incompetent, it is stripped at the same time from an incision posterior to the lateral malleolus to the popliteal space. After stripping the veins, the leg is held in the vertical position to permit broken vessel ends to retract, constrict, and clot. After the stripping procedure, collateral veins are removed by the avulsion-extraction technique. By working through small (5 to 8 mm) transverse incisions, segments of vein 10 to 20 cm long can be removed by dissecting subcutaneously along the vein with a hemostat, and then grasping, avulsing, and removing the vein. With practice, long segments of vein in all quadrants can be removed through these small incisions. Bleeding is controlled by elevation and pressure. As many as 40 incisions are made in severe cases, but their small size and transverse direction permit closure with a single suture. Before closure of the incisions, a rolled towel is rolled repeatedly from the knee to the ankle and from the knee to the groin to express any clots that may have accumulated. The groin incision is approximated with nylon mattress sutures and all other incisions are closed with a suture. To control bleeding, it is necessary to apply pressure to the limb. Typically, such pressure is applied by a gauze bandage wrap or support stocking for several weeks to several months after treatment.

Another common technique employed in the treatment of varicose veins involves the injection of a sclerosing agent to destroy a venous channel. In order for such sclerotherapy to be effective, it is necessary to apply pressure to the treated venous channel (after injection of the sclerosing agent) such that the blood does not pool or flow back into the treated venous channel. Typically, such pressure is applied by a gauze bandage wrap or support stocking for several days to several weeks after treatment.

As can be readily appreciated, it is cumbersome and difficult to apply pressure to the treated limb as part of these treatments. Hand pressure and towel wraps are effective in applying pressure to a limited part of the limb at any one time. Moreover, the person(s) exerting the pressure must be vigilant in covering the treated areas of the limb. Moreover, these methods fail to provide a mechanism to accurately control and monitor the amount of pressure applied to the treated limb.

Similarly, gauze wraps are cumbersome to use and fail to provide a mechanism to accurately control the pressure applied to the treated areas of the limb. It can therefore be appreciated that it would be desirable to provide a mechanism that can be used to accurately control and monitor the pressure applied to the treated areas of a human limb in conjunction with treatment of varicose veins in the limb.

### SUMMARY OF THE INVENTION

It is therefore an object of the invention to provide a mechanism that can be used to accurately control the pressure applied to the treated areas of a human limb in conjunction with treatment of varicose veins in the limb.

It is another object of the invention to provide a mechanism that can be used to accurately monitor the pressure applied to the treated areas of a human limb in conjunction with treatment of varicose veins in the limb.

It is a further object of the invention to provide a mechanism that can be used to apply, control and monitor pressure to the treated areas of a human limb in conjunction with treatment of varicose veins in the limb in a manner that requires minimal human intervention.

It is still another object of the invention to provide a mechanism that can be used to apply pressure to the treated areas of a human limb in conjunction with treatment of varicose veins in the limb in a manner that also provides stability to the treated limb. In accord with these objects, which will be discussed in detail below, a device is provided for applying pressure to the human thigh for use in conjunction with treatment of varicose veins of the human thigh. The device includes a flexible member and at least one air bladder chamber integral thereto that are adapted to securely wrap around the human thigh. A tube, in fluid communication with the air bladder chamber(s), extends to an air pumping mechanism that operates in response to manual manipulation to inflate the air bladder chamber(s) to a pressurized state. According to one embodiment of the invention, the flexible member preferably extends below knee joint level and is adapted to securely wrap around lower portions of a leg to provide stability to the leg. The flexible member includes an opening at the knee joint level to enable a patella to protrude therethrough.

It will be appreciated that when the flexible member and air bladder chamber(s) of the vein compressor mechanism are securely held over the treated areas of the thigh and the air bladder chamber(s) are inflated to the desired pressure, pressure is applied to the treated areas of the thigh. Such pressure is useful in conjunction with treatment of varicose veins in the thigh as described herein.

According to another embodiment of the invention, the device includes a flexible member and at least one air bladder chamber integral thereto that are adapted to securely wrap around the human lower leg. The air bladder chamber is substantially longer in a first dimension than in a second dimension orthogonal thereto such that the air bladder chamber can be positioned to cover a portion of the human lower leg that is relatively long in the vertical dimension and narrow in the horizontal dimension. A tube, in fluid communication with the air bladder chamber, extends to an air pumping mechanism that operates to inflate the air bladder chamber to a pressurized state. When the flexible member and air bladder chamber are securely held over the long narrow portion of the lower leg and the air bladder chamber is inflated to the desired pressure, local pressure is applied to the long narrow lower leg portion. Such local pressure is useful in conjunction with treatment of varicose veins in specific areas of the lower leg as described herein. Such a vein compressor mechanism is also readily adapted to provide local pressure to specific areas of the upper leg as well.

According to a preferred embodiment of the present invention, the air pumping mechanism of the device comprises a pumping bulb and valve that are manually manipulated to inflate the air bladder chamber(s).

According to yet another embodiment of the invention, the device includes a pressure gauge in fluid communication with the air bladder chamber(s), which provides a visual indication of pressure levels within the air bladder chamber(s).

Additional objects and advantages of the invention will become apparent to those skilled in the art upon reference to the detailed description taken in conjunction with the provided figures.

### BRIEF DESCRIPTION OF THE DRAWINGS

FIG. 1 is an isometric view of a vein compressor mechanism in accordance with the present invention, showing the mechanism securely wrapped around the thigh of a human patient.

FIG. 2 is an isometric view of the vein compressor mechanism of FIG. 1, showing the mechanism in its unwrapped state before it is securely wrapped around the thigh of a human patient.

FIG. 3 is an isometric view of a vein compressor mechanism in accordance with the present invention, showing the mechanism securely wrapped around the lower leg of a human patient.

FIG. 4 is an isometric view of the vein compressor mechanism of FIG. 2, showing the mechanism in its unwrapped state before it is securely wrapped around the lower leg of a human patient.

### DETAILED DESCRIPTION OF THE PREFERRED EMBODIMENTS

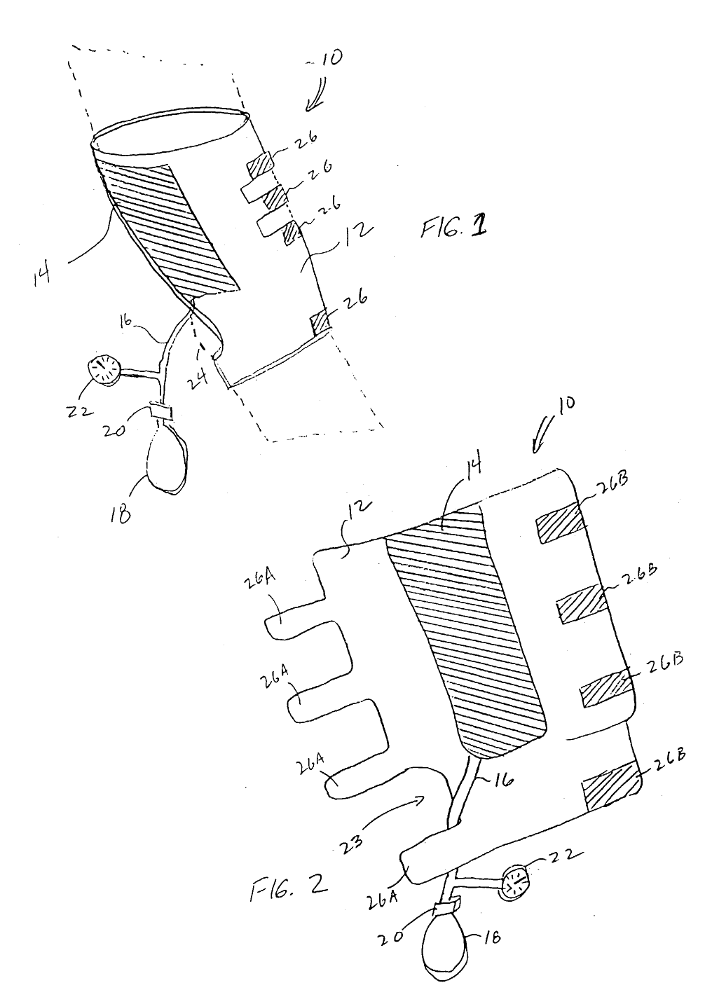

Turning now to FIGS. 1 and 2, a vein compressor mechanism is provided for applying pressure to the thigh of the human body for use in conjunction with treatment of varicose veins in the thigh. The vein compressor mechanism 10 includes a flexible member 12 having one or more inflatable air bladder chambers 14 (for example, one shown) integral thereto. Preferably, the flexible member 12 comprises two layers of elastomeric material, and the air bladder chamber(s) 14 are affixed between these two layers by nylon threads or other suitable fastening means. Alternatively, the flexible member 12 may include pockets into which the air bladder chamber(s) 14 are removably inserted and securely held therein. A tube 16, which is in fluid communication with the air bladder chamber(s) 14, extends to a pumping bulb 18. The pumping bulb 18, which is preferably made of rubber, includes a valve 20 that regulates the pumping of air into the air bladder chamber(s) 14 via the tube 16. Air is pumped into the air bladder chamber(s) 14 by squeezing the pumping bulb 18. In this manner, the air bladder chamber(s) 14 are placed into a pressurized state. Preferably, a pressure gauge 22 is operably coupled to the air bladder chamber(s) 14 to provide a visual indication of the pressure level therein. Preferably, the air bladder chamber(s) 14 are disposed along the inner portion of the thigh as shown, which enables the air bladder chamber(s) to apply pressure to the long saphenous vein of the patient when securely wrapped around the patient's thigh and inflated. However, the flexible member 12 and air bladder chamber(s) 14 may be adapted such that they are disposed along other portions of the upper leg (e.g., the portions of the leg at or above the knee), which enables the air bladder chamber(s) 14 to apply pressure to other portions of the upper leg when inflated. The flexible member 12 defines an opening 23 at the knee joint level to enable the patella (knee cap) 24 to protrude therethrough as shown. The flexible member 12 preferably extends below the knee joint level and securely wraps around portions of the lower leg as shown to provide stability to the leg.

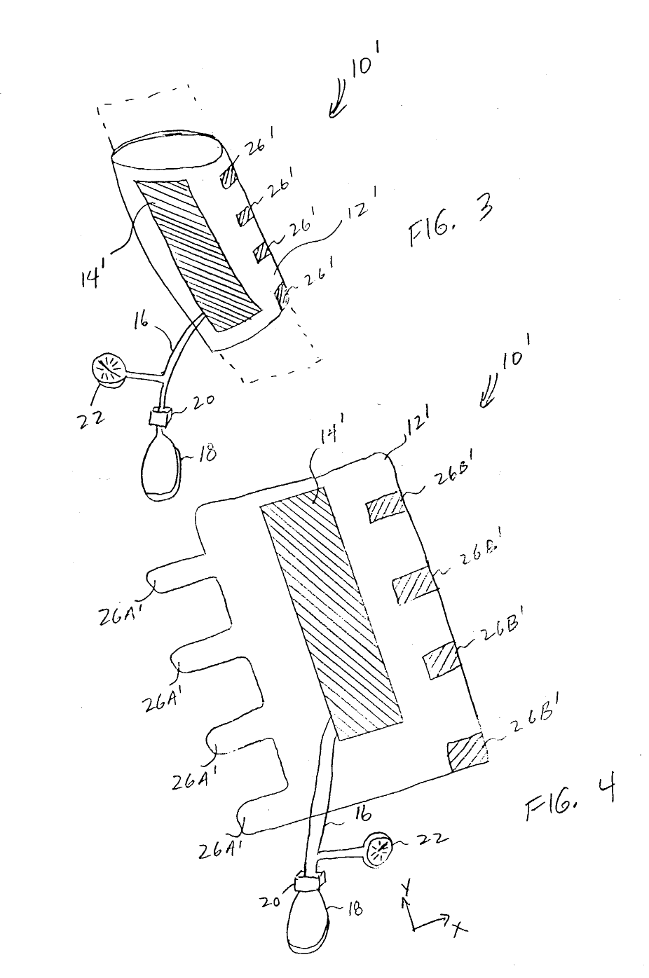

Turning now to FIGS. 3 and 4, a vein compressor mechanism is provided for applying pressure to the lower leg of the human body for use in conjunction with treatment of varicose veins in the lower leg. The vein compressor mechanism 10′ includes a flexible member 12′ having an inflatable air bladder chamber 14′ integral thereto. Preferably, the flexible member 12′ comprises two layers of elastomeric material, and the air bladder chamber 14′ is affixed between these two layers by nylon threads or other suitable fastening means. Alternatively, the flexible member 12′ may include a pocket into which the air bladder chamber 14′ is removably inserted and securely held therein.

Similar to the device of FIGS. 1 and 2, a tube 16 is provided which is in fluid communication with the air bladder chamber 14′ and extends to the pumping bulb 18. The pumping bulb 18, which is preferably made of rubber, includes a valve 20 that regulates the pumping of air into the air bladder chamber 14′ via the tube 16. Air is pumped into the air bladder chamber 14′ by squeezing the pumping bulb 18. In this manner, the air bladder chamber 14′ is placed into a pressurized state. Preferably, a pressure gauge 22 is operably coupled to the air bladder chamber 14′ to provide a visual indication of the pressure level therein.

The air bladder chamber 14′ is substantially longer in a first dimension (e.g., the Y dimension of FIG. 4) than in a second dimension orthogonal thereto (e.g., the X dimension of FIG. 4) such that the air bladder chamber 14′ can be positioned to cover a portion of the human lower leg that is relatively long in the vertical dimension and narrow in the horizontal dimension as shown in FIG. 3. Preferably, the air bladder chamber 14′ is disposed such that it covers the calf portion of the lower leg, which enables the air bladder chamber 14′ to apply pressure to the short saphenous vein of the patient when securely wrapped around the patient's lower leg and inflated. However, the flexible member 12′ and air bladder chamber 14′ may be adapted such that they are disposed along another portion of the lower leg (e.g., a portion of the leg below the knee), which enables the air bladder chamber 14′ to apply local pressure to such portion of the lower leg when inflated. Alternatively, the flexible member 12′ and air bladder chamber 14′ may be adapted such that they are disposed along a portion of the upper leg (e.g., a portion of the leg at or above the knee), which enables the air bladder chamber 14′ to apply local pressure to such portion of the upper leg when inflated.

Preferably, the flexible members of the vein compressor devices described above include multiple hook and loop enclosures 26 (labeled 26A and 26B in FIG. 2 and labeled 26A′ and 26B′ in FIG. 4). In the preferred embodiment, the flexible member includes four hook and loop enclosures as shown in FIGS. 1 through 4. These multiple enclosures enable the flexible member and the air bladder chamber(s) integrated thereto to be securely wrapped around a portion of the human leg.

If desired, other suitable fastening means may be used to secure the flexible member and the air bladder chamber(s) to the human leg. For example, the flexible member may be adapted to form a sleeve-like shape. In this configuration, the flexible member is slid over the patient's leg until it is disposed in the desired position.

Preferably, pressure in the air bladder chamber(s) is reduced/removed (e.g., the air bladder chamber(s) are deflated) by manipulation of the valve 20, and the vein compressor mechanism is removed from the leg by manually detaching the hook and loop closures and unwrapping the flexible member from around the leg.

The vein compressor mechanisms of FIGS. 1 through 4 are used in conjunction with treatment of one or more varicose veins (for example, the long saphenous vein of the thigh or the short saphenous vein of the lower leg) to apply pressure to such veins during treatment. Preferably, such pressure is applied by manipulating the pumping bulb 18 until the air bladder chamber(s) are inflated to a pressure on the order of 80 mmHg. Preferably, this target pressure level is visually indicated on the pressure gauge 22 of the vein compressor mechanism. When used in conjunction with vein ligation and/or surgical vein stripping, the pressure applied to the treated venous channels by the vein compressor mechanism of the present invention controls bleeding that results from this procedure. Also, when used in conjunction with sclerotherapy, the pressure applied to the treated venous channels by the vein compressor mechanism of the present invention significantly reduces the amount of blood that pools (or might potentially flow back) into the treated venous channel. This enables the sclerosing agent to have maximum effect in destroying the venous channel in the treated area of the thigh. It is contemplated that the patient may utilize the vein compressor device of FIGS. 1 through 4 to apply pressure to the treated areas of the leg for an extended period of time (e.g., periods of days/weeks) subsequent to treatment.

Advantageously, the vein compressor mechanism of the present invention provides for accurate control and monitoring of the pressure applied to the treated areas of a human leg in conjunction with treatment of varicose veins in the thigh. Furthermore, it limits the amount of human intervention involved in the application, control and monitoring of pressure with regard to the treated areas of a human leg.

There have been described and illustrated herein a preferred embodiment of a device (and corresponding method of operation) that is secured to a portion of the human leg and controlled to apply pressure to portions of the human leg for use in conjunction with treatment of varicose veins in the human leg. While a particular embodiment of the invention has been described, it is not intended that the invention be limited thereto, as it is intended that the invention be as broad in scope as the art will allow and that the specification be read likewise. Thus, while particular fastening mechanisms and particular pressure control mechanisms have been disclosed, it will be appreciated that other suitable mechanisms that may be used as well. For example, the air pump mechanism may include an automatic air pumping mechanism (typically actuated by an electric motor) rather than a hand-held manually actuated air pumping mechanism as described above. In addition, the air pump mechanism may be removably coupled to tubing that leads to the pressure valve and air chamber(s) of the device such that air pump mechanism can be disconnected from the device with the pressure valve closed (thereby maintaining the device in its pressurized state). Also, the vein compressor mechanism may extend to cover lower and/or higher portions of the leg than those shown. In addition, while the vein compressor device is preferably used in conjunction with vein ligation and/or surgical stripping procedures or sclerotherapy as described herein, it will be recognized that it can be used in conjunction with other treatments of varicose veins of the upper leg/lower leg as well as in conjunction with treatments of other venous diseases that afflict the upper leg/lower leg. It will therefore be appreciated by those skilled in the art that yet other modifications could be made to the provided invention without deviating from its spirit and scope as claimed.

What is claimed is:

1. A device for applying pressure to the human thigh comprising:
a) a flexible member and at least one air bladder chamber integral thereto that are adapted to securely wrap around the human thigh, wherein said flexible member includes an opening that enables a patella to protrude therethrough;
b) a tube, in fluid communication with said at least one air bladder chamber;
c) an air pumping mechanism coupled to said tube and that operates to inflate said at least one air bladder chamber to a pressurized state.

2. The device according to claim 1, wherein:
said flexible member is adapted to extend below knee joint level and securely wrap around a lower portion of a leg to provide stability to said leg.

3. The device according to claim 1, wherein:
said air pumping mechanism comprises a pumping bulb and valve that are manually manipulated to inflate said at least one air bladder chamber to said pressurized state.

4. The device according to claim 3, wherein:
said valve is adapted to be manually manipulated to deflate said at least one air bladder chamber to an unpressurized state.

5. The device according to claim 1, further comprising:
a pressure gauge, in fluid communication with said at least one air bladder chamber, which provides a visual indication of pressure levels within said at least one air bladder chamber.

6. The device according to claim 1, further comprising:
a plurality of hook and loop closures, affixed to said flexible member, that are joined together to securely wrap said flexible member around the human thigh.

7. The device according to claim 1, wherein:
said at least one air bladder chamber is disposed such that it covers at least one venous channel that is subject to medical treatment.

8. The device according to claim 7, wherein:
said at least one venous channel includes portions of the long saphenous vein.

9. A method of treating one or more varicose veins in the thigh of a human patient, the method comprising the steps of:
a) providing a device for applying pressure to the thigh, said device comprising
i) a flexible member and at least one air bladder chamber integral thereto that are adapted to securely wrap around the thigh, wherein said flexible member includes an opening to enable a patella to protrude therethrough,
ii) a tube, in fluid communication with said at least one air bladder chamber, and
iii) an air pumping mechanism coupled to said tube and that operates to inflate said at least one air bladder chamber to a pressurized state;
b) manipulating said flexible member such that said flexible member and said at least one air bladder chamber integral thereto are securely wrapped around the human thigh, whereby the patella of knee of the human patient protrudes through said opening in said flexible member; and
c) inflating said at least one air bladder chamber to a pressurized state with said air pumping mechanism.

10. A method according to claim 9, further comprising:
manipulating said flexible member such that said flexible member is securely wrapped around a lower portion of a leg of the human patient below the thigh to provide support to the leg of the human patient.

11. A method according to claim 9, wherein:
said air pumping mechanism comprises a pumping bulb and valve, and said inflating step c) comprises manually manipulating said pumping bulb and valve to inflate said at least one air bladder chamber to said pressurized state.

12. The method according to claim 11, further comprising the step of:
manually manipulating said valve to deflate said at least one air bladder chamber to an unpressurized state.

13. The method according to claim 9, wherein:
said pressurized state is approximately 80 mmHg.

14. The method according to claim 9, wherein:
said device includes a pressure gauge in fluid communication with said at least one air bladder chamber, which provides a visual indication of pressure levels within said at least one air bladder chamber.

15. The method according to claim 9, wherein:
said device includes a plurality of hook and loop closures, affixed to said flexible member, that are joined together to securely wrap said flexible member around the human thigh, and said step b) comprises manually joining said plurality of hook and loop enclosures to securely wrap said flexible member around the human thigh.

16. The method according to claim 9, further comprising the step of:
positioning said at least one air bladder chamber such that it covers at least one venous channel that is subject to medical treatment.

17. The method according to claim 16, wherein:
said at least one venous channel includes portions of the long saphenous vein of a leg of the human patient.

18. A device for applying pressure to a portion of the human leg comprising:
a) a flexible member and an air bladder chamber integral thereto that are adapted to securely wrap around the human leg, wherein said air bladder chamber is substantially longer in a first dimension than in a second dimension orthogonal thereto such that the air bladder chamber can be positioned to cover a portion of the human leg that is relatively long in the vertical dimension and narrow in the horizontal dimension;
b) a tube, in fluid communication with said air bladder chamber; and
c) an air pumping mechanism coupled to said tube and that operates to inflate said air bladder chamber to a pressurized state.

19. The device according to claim 18, wherein:
said air pumping mechanism comprises a pumping bulb and valve that are manually manipulated to inflate said air bladder chamber to said pressurized state.

20. The device according to claim 19, wherein:
said valve is adapted to be manually manipulated to deflate said air bladder chamber to an unpressurized state.

21. The device according to claim 18, further comprising:
a pressure gauge, in fluid communication with said air bladder chamber, which provides a visual indication of pressure levels within said air bladder chamber.

22. The device according to claim 18, further comprising:
a plurality of hook and loop closures, affixed to said flexible member, that are joined together to securely wrap said flexible member around the human leg.

23. The device according to claim 18, wherein:
said air bladder chamber is disposed such that it covers at least one venous channel that is subject to medical treatment.

24. The device according to claim 23, wherein:
said at least one venous channel includes portions of the short saphenous vein.

25. A method of treating one or more varicose veins in the leg of a human patient, the method comprising the steps of:
a) providing a device for applying pressure to the human leg, said device comprising
i) a flexible member and an air bladder chamber integral thereto that are adapted to securely wrap around the human leg, wherein said air bladder chamber is substantially longer in a first dimension than in a second dimension orthogonal thereto such that the air bladder chamber can be positioned to cover a portion of the human leg that is relatively long in the vertical dimension and narrow in the horizontal dimension,
ii) a tube, in fluid communication with said air bladder chamber, and
iii) an air pumping mechanism coupled to said tube and that operates to inflate said air bladder chamber to a pressurized state;
b) manipulating said flexible member such that said flexible member and said air bladder chamber integral thereto are securely wrapped around the human leg; and
c) inflating said at least one air bladder chamber to a pressurized state with said air pumping mechanism.

26. A method according to claim 25, wherein:
said air pumping mechanism comprises a pumping bulb and valve, and said inflating step c) comprises manually manipulating said pumping bulb and valve to inflate said air bladder chamber to said pressurized state.

27. The method according to claim 26, further comprising the step of:
manually manipulating said valve to deflate said air bladder chamber to an unpressurized state.

28. The method according to claim 25, wherein:
said pressurized state is approximately 80 mmHg.

29. The method according to claim 25, wherein:
said device includes a pressure gauge in fluid communication with said air bladder chamber, which provides a visual indication of pressure levels within said air bladder chamber.

30. The method according to claim 25, wherein:
said device includes a plurality of hook and loop closures, affixed to said flexible member, that are joined together to securely wrap said flexible member around the human leg, and said step b) comprises manually joining said plurality of hook and loop enclosures to securely wrap said flexible member around the human leg.

31. The method according to claim 25, further comprising the step of:
positioning said air bladder chamber such that it covers at least one venous channel that is subject to medical treatment.

32. The method according to claim 31, wherein:
said at least one venous channel includes portions of the short saphenous vein of a leg of the human patient.
